(19) United States
(12) Patent Application Publication
(10) Pub. No.: US 2005/0090823 A1
(43) Pub. Date: Apr. 28, 2005

(54) Posterior fixation system

(76) Inventor: Christopher Shawn Bartimus, Payette, ID (US)

Correspondence Address: Chris Bartimus, 1020 N. 2nd St., Payette, ID, 83661, US

(21) Appl. No.: 10/695,586

(22) Filed: Oct. 28, 2003

(51) Int. Cl. A61F 5/04; A61B 17/56; A61F 2/30

(52) U.S. Cl. 606/61; 606/60

(57) ABSTRACT

A posterior fixation system for the human spine associated with fusion or corrective procedures in achieving proper alignment of the spine with regard to the patient's natural or corrective posture through optional postoperative re-adjustment. The fixation system may include a clamping block secured to a bone segment by pedicle screws or other anchoring device whereby a cross member passes through the multi-axial clamping block and is attached to a bone segment opposite the bridged section. The cross member is clamped and locked into position by engaging the set screw which is also accessible postoperatively by non-invasive means. The fixation of the bone segments promotes healing over an affected area while facilitating a proper alignment of the spine.

### BACKGROUND OF INVENTION

Spinal fixation and instrumentation is a well-accepted practice for correcting spinal deformities and other injuries. It is often used in conjunction with inter-vertebral fusion and various fusion devices and methods to facilitate the fixation necessary for the fusion resulting in a successful and anatomically correct fusion.

This spinal instrumentation is often attached to pedicle screws, screws, hooks and clamps which are imbedded or affixed to various spinal bone segments.

Most spinal instrumentation would fall under the category of either anterior or posterior. Anterior instrumentation would be installed from the front or onto the front of the spine therefore requiring a low, flat profile on the surface of the spine and having a very difficult means to re-adjust without invasive surgery. Some in the industry may prefer the anterior approach on certain areas of the spine if it is surgically feasible because the results of an anatomically correct fusion are believed to be higher due to the relatively flat surfaces to mount to. There is usually no need for disruptive posterior instrumentation on the back of the spine using the anterior approach.

Posterior instrumentation although having a greater challenge of traversing the difficult terrain and anchoring solutions can be less invasive along with having greater possibilities of using various procedures and methods and the number of times they can be performed without adverse affects. Spinal instrumentation in conjunction with fusion is usually left in place permanently although it is generally used to facilitate the fusion process and no longer necessary after a successful fusion.

One goal of spinal instrumentation apart from providing stability at the fusion site promoting healing is the corrective alignment of the spine to receive the axial loads and others stresses that are normally placed upon it thereby restoring the individual to as great of physical health as possible. It is not uncommon for individuals who have had fusion at a level to experience problems over time and require fusion on adjacent levels. The disks at adjacent levels and subsequent levels are often required to absorb differences in alignment put upon them by the change in alignment by the spinal instrumentation. It is often a surgeon's best guess from x-ray images taken at the time of surgery that determine the outcome of the patient's alignment. One difficult factor in estimating this alignment of the spine is the patients posture, deformities and otherwise individual attributes idealistic or not. Another factor weighing against a perfect or natural alignment can be the instrumentation itself, either by the limitations of the component or the angles of the of pedicle screws they attach to or a combination of both. Due to the nature of the surgical environment and time frame it must be completed adds to the challenge also. Once a pedicle screw has been set it is committed and with any success within the limitations of the fixation component with respect to the desired alignment. With all these factors and others to overcome there remains one more, fine adjustment. It is possible that even small adjustments in these axial angles can make or break the success rate especially in the long term. Most current instrumentation seems to lack somewhat in this versatility and accuracy leaving the success rate leaning heavily on the skill of the surgeon and chance.

Several recent fixation devices have expanded the multi-axial and linear adjusting capabilities. Such examples are demonstrated in U.S. Pat. Nos. 5,797,911, 5,954,725, and 6,485,491. These constructs add a margin of error in the precision necessary when anchoring the devices to achieve a desired alignment but may not address all possibilities especially over a single level or each level independently. Examples of axial and linear movements taken from an upright spinal perspective may include but not limited to, forward and backward axial rotation, right to left axial rotation, rotation of the spinal axis, forward and backward horizontal linear movement, right to left horizontal linear movement, and up and down vertical linear movement. The up and down vertical movement or expansion of the disk space is especially helpful overcoming obstruction to movement by an inter-vertebral fusion device and the possibility of correcting it by non-invasive methods and procedures. A visual reference of the axial and linear movements mentioned above and addressed in the present invention are shown in FIG. 13.

It is therefore desirable for a fixation system to have the ability to be minutely adjustable in all necessary axial and linear movements with a wide range of limitation. A wide range of limitation would allow for a greater margin of error in the installation of supporting pedicle screws or components raising the success rate for surgeons who may not have the latest high tech advantages at their disposal. It is also desirable to be able to make those adjustments postoperatively and by doing so the patient can have input as to that sense of balance and posture. The patient is able to experience the posture given from the time of surgery and if not satisfied with it the physician can loosen the clamping mechanism temporarily for the patient to make a correction in posture and/or relieving any stresses upon the spine, all done within a limited time frame of the fusion process. The loosening of the clamping mechanism could be done by a non-invasive procedure with a local anesthetic.

All of the axial and linear movements mentioned are addressed in the mechanism directly or in combination with the pedicle screws. The alignment of the upper and lower vertebra in the right to left horizontal plane are for the most part set at the time of surgery when the nuts on the pedicle screws are secured. When used in pairs, one on each side as is the general practice, the amount of adjustment left in that direction of movement would then be minimal. There is however enough movement to relieve a little pressure in any direction but restrictive enough to add a safety factor against lateral shifting. This restrictive behavior would be considered an important feature to avoid disturbing the placement of the fusion device or causing injury.

This fixation system may be considered a little bulky in size compared to some prior art but there are measures that can be taken to minimize discomfort that could arise. There are at least three factors that could help eliminate discomfort. The exterior shape of the component's are rounded off to eliminate sharp edges that come in contact with various tissues. The top of the clamping block is flat and rounded which may be much more acceptable to the muscle groups and tissues above it contrasting the often narrow and pointed shape of some instrumentation. The fixation components could be set lower into the area of the fusion by removing the unnecessary bone structures that may hold them out if the components are to be left in permanently. Once fusion has taken place and the fixation components are now longer necessary it may be feasible to remove them either by peeling back the muscle from the bone segments of the spine or by offsetting the fixation components to the outside of the spinal axis and removing them through an incision in the muscles and tissues above them.

The composition of the fixation components when used inside the body should meet requirements for inter-body compositions for example titanium used for its high strength to weight ratio, non-corrosive and non-magnetic characteristics, but is not considered limited to such. This fixation system is not considered to be limited in scope and location of use inside or outside the body or for other mechanical purposes. It is within the scope of the invention to be useable in series for multiple fusions at several levels at once and scalable to appropriate sizes and stresses. It is not considered limited to inter-vertebral fusion but may be used for corrections and injuries such as scoliosis and fractures.

### SUMMARY OF INVENTION

A form of the unique spinal fixation system significantly reduces the limitations and shortcomings of the current methods and instrumentation and offers additional advantages and methods as aforementioned.

The present invention is directed to a method of installation that is partially established in the field but add features and capacities that further enhance and simplify the process. The surgical procedure is similar to that being used by prior art accept for the simplified method of locking the device in a preferred alignment having accessibility to that adjustment by non-invasive, postoperative means. The present invention, in a certain embodiment, is directed to a typical but simplified method for posterior spinal fixation, comprising (a) preparing the location for the embodied mechanism by removal of bone or unnecessary obstructions for the desired positioning, (b) placing the embodied mechanism in the prepared location and drilling the pilot holes for the pedicle screws within the alignment limitations of the embodied mechanism using the holes in the connectors of the embodied mechanism as a guide for angles and placement, (c) affixing pedicle screws or anchoring device as presently used in the field to each side of the disk space to be bridged, (d) attaching the embodied mechanism on the threaded pedicle screws with nuts, (e) aligning the spine across the affected disk space to be anatomically correct and tightening the set screw and, (f) making adjustments postoperatively by a non-invasive procedure if necessary.

This embodiment is not limited to single level bridging or fusion but can be assembled in series for multiple levels with planning of the location, angles and specifications of the connectors of the embodied mechanism.

The present invention, in an alternate embodiment, may utilize the same but expanded clamping block with two or more cross members secured therein. For example, this alternate embodiment may have a cross member extending out from the same side or opposite sides of the clamping block. In this embodiment the clamping block may or may not have a connector attached to the clamping block and connectors attached to one end of the cross members unless otherwise used in conjunction with other devices. With a connector attached to the clamping block and two opposing cross members the embodied mechanism could be anchored to three different vertebra, bridging two affected disks, utilizing the same clamping block.

### BRIEF DESCRIPTION OF DRAWINGS

Further advantages of the present invention will become more apparent with the assistance of the following descriptions of the preferred embodiment and the detailed drawings.

FIG. 1 depicts a perspective of prior art.

FIG. 2 depicts a perspective and overview of an embodiment of a fixation system.

FIG. 3 depicts a top view of a fixation system showing cross sectional lines for FIG. 5 and FIG. 8.

FIG. 4 depicts side view of a fixation system.

FIG. 5 depicts a cross sectional side view of a fixation system.

FIG. 6 depicts a side view of a fixation system.

FIG. 7 depicts a side view of a fixation system.

FIG. 8 depicts a cross sectional side view of a fixation system.

FIG. 9 depicts a side view of a fixation system.

FIG. 10 depicts a top view of a top view in a pictorial representation of the lower spine instrumented with the fixation system in one embodiment of the present invention.

FIG. 11 depicts a perspective view of a fixation system attached by pedicle screws.

FIG. 12 depicts a perspective view of an alternate fixation system showing a double cross member embodiment attached in series with a single cross member embodiment.

FIG. 13 and a visual reference of the axial and linear movements that are under consideration by the present invention.

The present invention shown forth in the drawings and described in detail although may be representative of a preferred embodiment demonstrating some of the inventions intended uses and configurations they are not intended to demonstrate all the possibilities or configurations within the scope and spirit of the invention as established in the claims.

### DETAILED DESCRIPTION

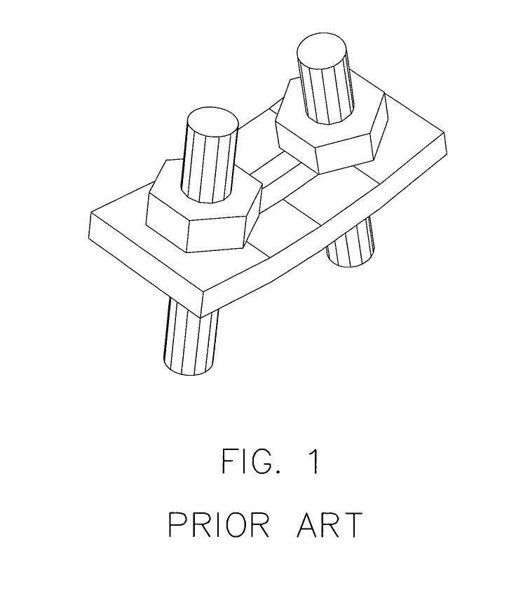

FIG. 1 refers to a perspective view of prior art showing a typical single level fixation system.

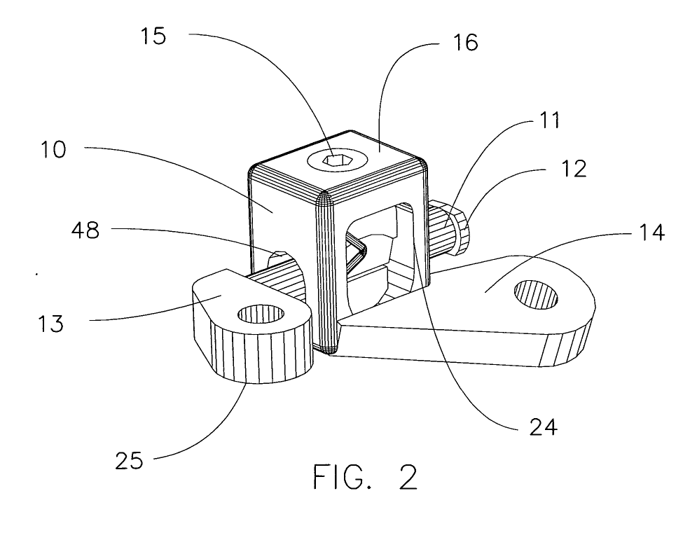

FIG. 2 refers to a perspective view of an embodiment of a fixation system showing the clamping block 10 and the set screw 15 center mounted in the clamping block. Visible in this view is a cross member 11, a cross member safety stop 12 attached to end of the cross member 11 to prevent the possibility of disassembling during installation. Clearly seen in this view is a clamping block connector 14 attached to the clamping block 10 and a cross member connector 13 attached to the cross member 11. Another feature visible in this view is the flat top with rounded sides 16 reducing any discomfort the recipient might experience.

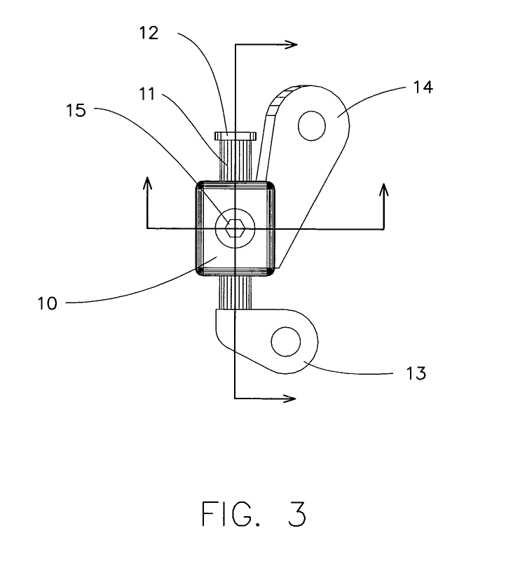

FIG. 3 refers to a top view of the preferred embodiment shown in FIG. 2. This embodiment shows the set screw 15 placed center in the clamping block 10. Also shown are cross sectional lines corresponding to the sectional views of FIG. 5 and FIG. 8.

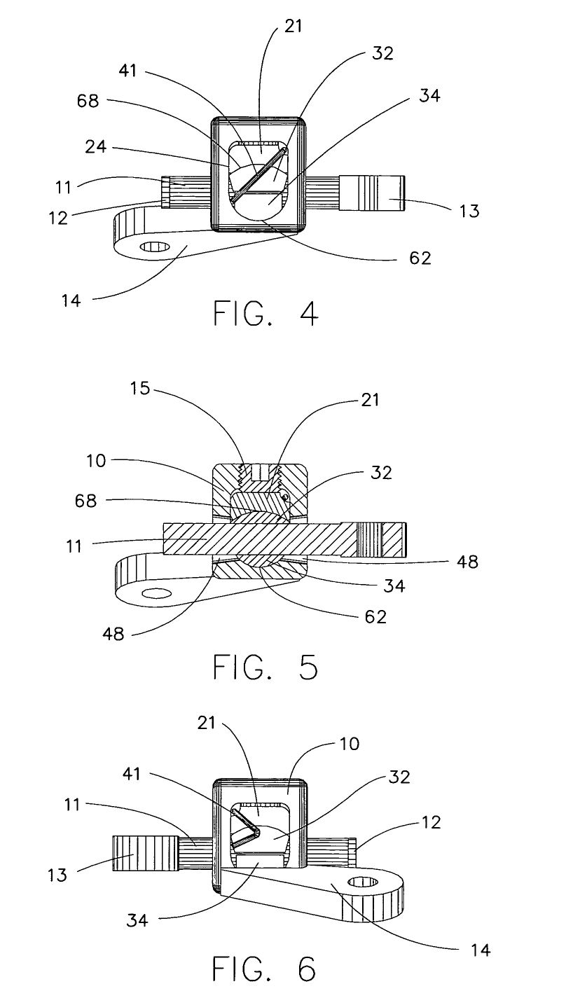

FIG. 4 refers to a side view of a preferred embodiment shown in FIGS. 2 and 3. This figure shows some of the mechanical workings specifically the thrust cap 21 which is held in position by the retaining wire 41, demonstrating one method of final assembly of the construct after sterilization yet can be easily disassembled and sterilized again if a different model of the device was selected after exposing it to a contaminating environment. Beneath the thrust cap 21 is the upper intermediary portion 32, from hereon referred to as upper portion 32, and is constructed to match the cylindrical arc of the thrust cap 21 and the cylindrical contour of the cross member 11 below it. At the bottom of the clamping block 10 is the lower intermediary portion 34, from hereon referred to as a lower portion 34 and is constructed to match the cylindrical arc of the bottom of the clamping block 10 and the contour of the cross member 11 above it. The construct may be made of any acceptable material such as titanium or an alloy thereof and the finish polished. It is most desirable to have the surface finish of the portions and their respective counterparts polished to at least a 1000 grit for minute adjustment and also when using titanium as the galling nature of titanium may cause a seizing of the parts after an initial tightening of the mechanism. Surface finishes may vary from smooth to very fine interconnecting grooves depending on material and application. Another notable feature is the openness of the construct 24 reducing the possibility of trapped air and body fluids, an important element to devices implanted in the body for safety and health reasons.

FIG. 5 refers to a cross sectional view of the preferred embodiment showing a set screw 15 set to apply pressure upon the thrust cap 21, the upper portion 32, the cross member 11, the lower portion 34 and onto the bottom of the clamping block 10. The center of the arc of the upper portion 68 is in opposition to the center of the arc of the lower portion 62, prohibiting the two arcs from having a central pivotal point, hence they cannot simply rotate like they would if the arcs formed a perfect circle. Friction between the parts at arcs 68 and 62 play a big part in this resistance to rotate but the flatter arc 68 forces the upper portion 32 to pick up additional friction from the cross member 11. Under stress to rotate the upper portion 11 would be forced to slide on the cross member 11 thus picking up more friction. This acts as a lock to prevent axial movement of the cross member 11 with respect to arcs of the upper 32 and lower 34 portions. There are other construction materials and types of surface finishes that it may serve better to have a flatter arc at the lower portion arc 62 like that of the upper portion arc 68. An example of such a material might be a stainless steel. Such an arc is not shown in this view but does not limit the preferred embodiment to the arcs shown. The smaller radius arc in the lower portion 62 as shown in this view is done so for ease in machining the clamping block 10 but a larger radius arc may be achieved by employing other manufacturing processes.

The clamping block 10 has an opening 48 on each side to receive the cross member 11. The opening angle shown demonstrates a cross member angle change of plus or minus 7.5 degrees but is not considered and absolute limitation upon which the device can be designed to go.

FIG. 6 refers to a side view of a preferred embodiment showing the opposite end of the retaining wire 41. It is bent in this fashion to counter pivoting in the clamping block and for ease of installation. Once the thrust cap 21 is slid into place over the upper portion 32 it would slide out the other side except for the retaining wire 41 keeping it located over the upper portion 32. This retaining wire 41 is the last step in assembly that keeps the unit from disassembling in the loosened state.

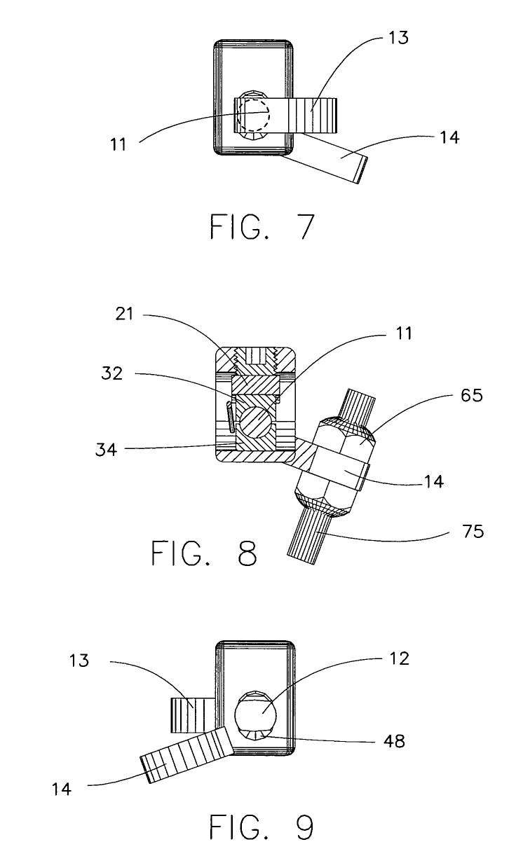

FIG. 7 Seen in this view from the side is a cross member connector 13 centered with the axis of the cross member 11. This attachment configuration may be moved up or down vertically in this view to accommodate using the components in series. The same would be true for the clamping block connector 14 which can be attached anywhere on the clamping block 10 to accommodate anchoring with respect to the varied angles and placement of pedicle screws 63 and other anchoring devices into bone segments. This versatility of angles is necessary as the options for drilling and anchoring the pedicles screws 63 vary from vertebra to vertebra up and down the spine.

FIG. 8 refers to a cross sectional view of the preferred embodiment showing the cylindrical shape of the cross member 11. The cylindrical shape of the cross member 11 allows the cross member to rotate. The in and out sliding movement and the rotation allows for forward and backward horizontal movement and rotation of the spinal axis. These basically align the adjacent vertebra and are for the most part set at the time of surgery once the nuts on the pedicle screws 62 are secured along with the added factor of a fusion device pressed between the vertebra. Another advantage to using the cylindrical shape is the ability to overcome differing angles of the pedicle screws 63 in that plane. Other non-cylindrical shapes may be used when it is not desirable for this added axis of movement. Adjustment of the fusion device can still me done postoperatively by non-invasive means because the present invention can be loosened allowing the disk space to be spread apart.

The clamping block connector 14 is shown at an incline to accommodate the pedicle screw 75 passing through a hole 93 in the clamping block connector 14 being anchored in a bone segment. If the ideal angle for the pedicle screw 75 is not achieved the adverse effect would only be that the angle of access to the set screw may be slightly off perpendicular with the exterior surface of the back. A nut 65 secures the clamping block connector 14 to the pedicle screw 75

FIG. 9 shows the cross member safety stop 12. The cross member is inserted through the opening 48 by turning it 90 degrees.

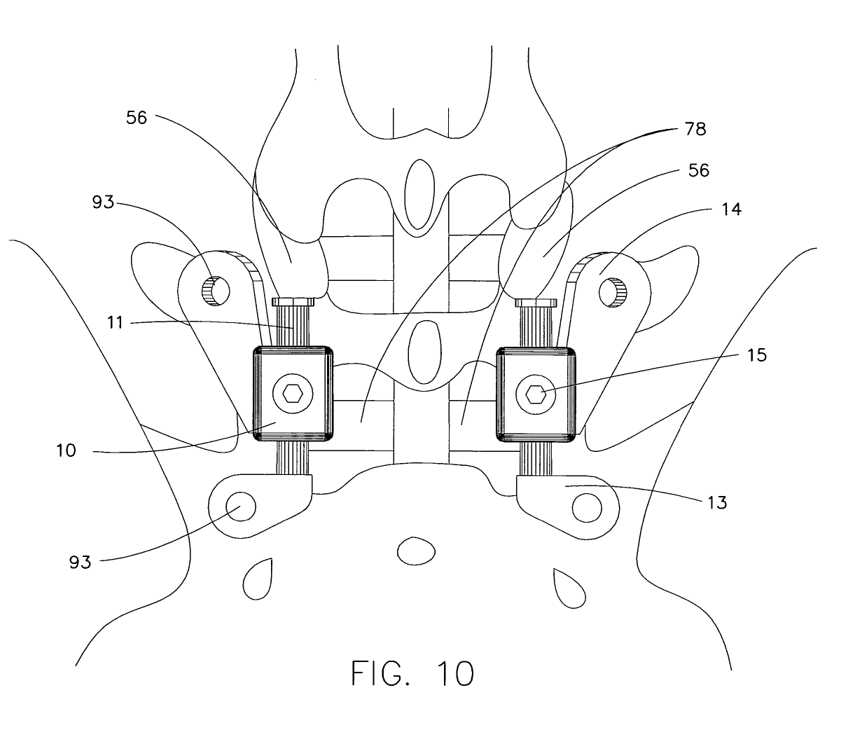

FIG. 10 refers to a pictorial top view of the preferred embodiment in position on a spinal column. At this level the present invention is configured so as not to disrupt the movement of the ligaments 56 of the next level up the spine. The cross members 11 should be installed close to parallel with the axis of the spine. This ensures the best adjustability. The clamping block connector 14 and the cross member connector 13 have holes 93 for mounting to pedicle screws 75 or other anchoring devices.

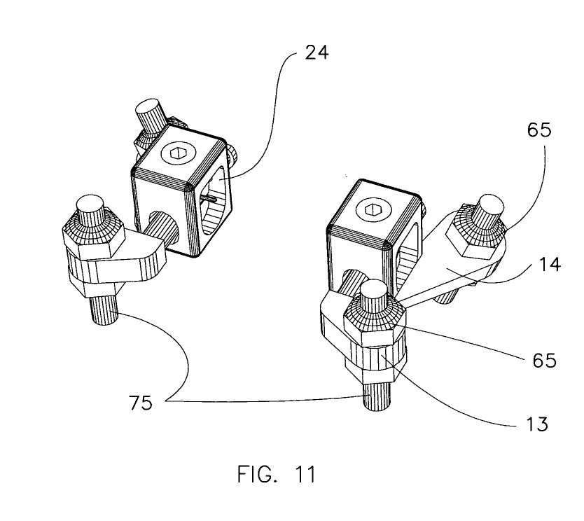

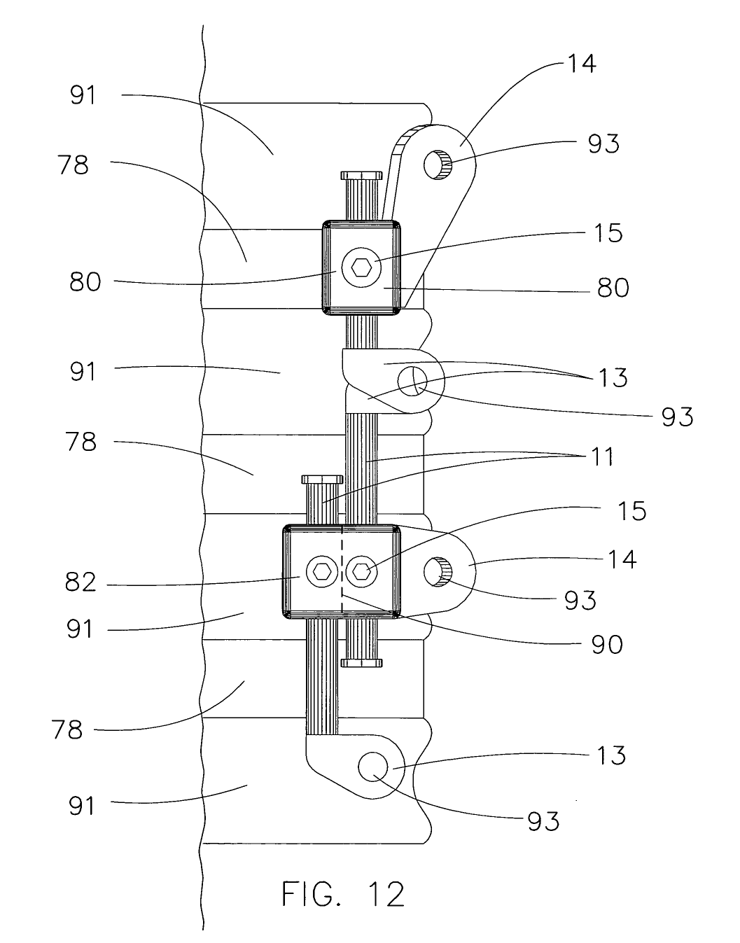

FIG. 11 is a perspective view showing the preferred embodiment attached to a threaded pedicle screw 72 with a nut 75.

FIG. 12 refers to an alternate preferred embodiment utilizing a double cross member unit 82, and a single cross member unit 80, attached in series to bridge three affected disk spaces 78. The double cross member unit 82 in this embodiment is equipped with two independent set screws 15 for maximum clamping pressure. A thin metallic separator 90 may be required between the two sides of a double cross member unit 82 to prevent minor overlapping and jamming of the independent sides. The holes 93 are for mounting to pedicle screws 75 which are embedded in bone segments 91.

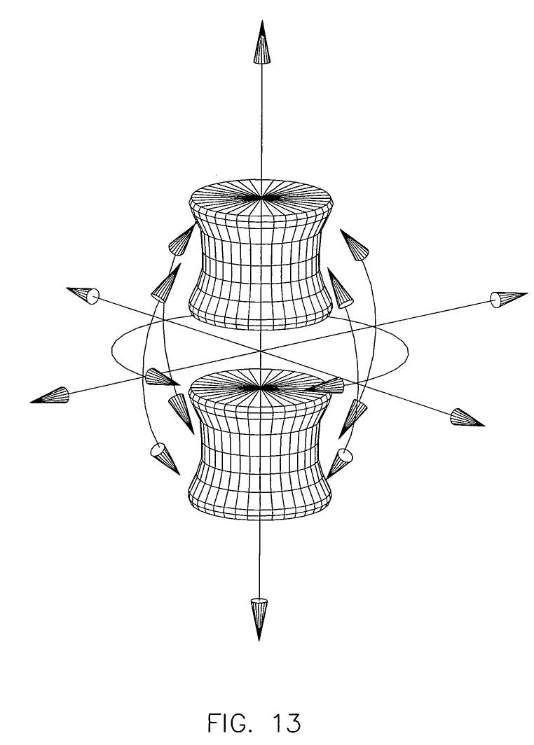

FIG. 13 refers to a visual reference of the axial and linear movements that are under consideration by the present invention taken from a patient's standing position are the forward and backward axial rotation, right to left axial rotation, rotation of the spinal axis, forward and backward horizontal linear movement, right to left horizontal linear movement, and up and down vertical linear movement.

Example sizes of the preferred embodiment of the components of this invention are listed in the following table, designated for the lumbar region in a full grown male. The specifications shown here are for example only as a patient's size and weight, the location implanted in the body, and the materials and process used to manufacture the device are all factors determining these specifications.

| | FIGURE | | | | | |
|---|---|---|---|---|---|---|
| | Call | Length | Width | Diameter | Radius | Depth |
| Component | Out | (in) | (in) | (in) | (in) | (in) |
| Clamping Block | 10 | .625 | .5 | NA* | NA | .73 |
| Cylindrical | 62 | .5 | .375 | .375 | .1875 | NA |
| segment | | | | | | |
| Cross member | 11 | 1.1 | NA | .2 | NA | NA |
| Cross member | 12 | .26 | .2 | .2 | .1 | .04 |
| safety stop | | | | | | |
| Thrust cap | 21 | .365 | .3 | NA | .5625 | .177 |
| Upper portion | 32 | .356 | .25 | .2 | .5625 | .168 |
| Lower portion | 34 | .312 | .25 | .2 | .1875 | .168 |
| Clamping block | 14 | 1.18 | .5 | .19 Hole | NA | .18 |
| connector | | | | | | |
| Cross member | 13 | .65 | .41 | .19 Hole | NA | .22 |
| connector | | | | | | |
| Set screw | 15 | .167 | NA | .25 | NA | .125 hex |

*NA = Not Applicable

The invention has been illustrated and described in the drawings and detailed its unique capabilities and should not be considered restricted from modifications, changes, or additions that come within the scope and spirit of the invention as defined by the following claims.

1. A multi-axial fixation device for use in aligning spinal members during operation, said device comprising:
a clamping block said clamping block defining a passageway, said passageway configured to receive a cross member therein, said clamping block having a clamping block connector connected thereto, said clamping block connector configured for connection with a first spinal member;
said cross member configured for insertion within said passageway, said cross member having a cross arm connector connected thereto, said cross arm connector configured to connect with a second spinal member, said cross member configured for variable locked positioning within said clamping block by a locking device;
said locking device configured to allow said cross member to be variously adjustably positioned and held within said clamping block;
said clamping block, said locking device and said cross member configured to allow variable positioning and locking of said cross member thereby allowing multiple adjustable positioning of said spinal members.

2. The multi-axial fixation device of claim 1 wherein said locking device comprises a plurality of stacked portions that are configured to lock and hold said cross member by compressive force upon said stacked portions.

3. The multi-axial fixation device of claim 1 wherein said passageway is a transverse multi-positional opening through said clamping block.

4. The multi-axial fixation device of claim 1 wherein said a cross arm connector and said clamping block connector are each configured to connect with a spinal member through the use of a pedicle screw.

5. The multi-axial fixation device of claim 1 wherein said cross member connector further comprises a safety stop; said safety stop configured to prevent withdrawal of said cross member from said passageway.

6. The multi-axial fixation device of claim 1 wherein said locking device is multiply adjustable through the use of a set screw.

7. The multi-axial fixation device of claim 6 wherein said clamping block further defines an aperture configured to maintain and hold said set screw within said clamping block.

8. The multi-axial fixation device of claim 1 wherein said clamping block is configured to have a flat surface with rounded edges to prevent discomfort that may be experienced by the recipient.

9. The multi-axial fixation device of claim 8 wherein said clamping block is configured to define at least one access opening said access opening configured to allow the passage of body fluids around said clamping block.

10. The multi-axial fixation device of claim 9 further comprising a retaining clip, said retaining clip configured to allow said locking device to be retained and removed from within said clamping block.

11. The multi-axial fixation device of claim 1 wherein said locking device comprises: a thrust cap, an upper portion and a lower portion, said thrust cap said upper portion and said lower portion each having a radius configured to interact with an adjoining portion when placed in a desired position and orientation, said upper portion and said lower portion each having a radius center and configured so that said radius centers are offset when said upper portion and said lower portion are placed in correct alignment within said clamping block.

12. The multi-axial fixation device of claim 11 wherein said cross member passes through said passageway and said locking device is configured to allow variable linear, rotational, and angular positioning of said cross member.

13. The multi-axial fixation device of claim 11 wherein said thrust cap is held in position by a retaining wire configured to retain said thrust cap from sliding out of said clamping block and preventing the whole system from disassembling when in a loosened state.

14. The multi-axial fixation device of claim 13 wherein said cross member further comprises a cross member safety stop, said cross member safety stop configured to prevent the cross member from disassembling, when said cross member is in a loosened state.

15. The fixation device of claim 1, wherein the angles and location of the cross member connector and the clamping block connector are configured for use in series and combination for use in multiple level fusions and bridging.

16. The fixation device of claim 1, wherein said locking device is configured for post operative access by a non-invasive means to accomplish a variety of post operative adjustments.

17. The fixation device of claim 16 wherein said device is configured to allow selective disengagement and adjustment of said spinal members in one direction while retaining said spinal members in a secured position in a second direction.

18. An improved method for posterior spinal fixation comprising:
preparing the location for the embodied mechanism by removal of bone or unnecessary obstructions for the desired positioning;
placing a fixation device in the prepared location and drilling the pilot holes for the pedicle screws within the alignment limitations of the fixation device using the holes in the connectors of the embodied mechanism as a guide for angles and placement, said fixation device having a clamping block said clamping block defining a passageway, said passageway configured to receive a cross member therein, said clamping block having a clamping block connector connected thereto, said clamping block connector configured for connection with a first spinal member, said cross member configured for insertion within said passageway, said cross member having a cross arm connector connected thereto, said cross arm connector configured to connect with a second spinal member, said cross member configured for variable locked positioning within said clamping block by a locking device, said locking device configured to allow said cross member to be variously adjustably positioned and held within said clamping block, said clamping block, said locking device and said cross member configured to allow variable positioning and locking of said cross member thereby allowing multiple adjustable positioning of said spinal members.
affixing pedicle screws or anchoring device as presently used in the field to each side of the disk space to be bridged,
attaching the embodied mechanism on the threaded pedicle screws with nuts;
aligning the spine across the affected disk space to be anatomically correct and tightening the set screw; and
making adjustments postoperatively by a non-invasive procedure if necessary.
